(19) United States
(12) Patent Application Publication
(10) Pub. No.: US 2022/0125889 A1
(43) Pub. Date: Apr. 28, 2022

(54) COMPOSITION COMPRISING ALBUMIN-COUPLED SLIT3 LRRD2 FOR PREVENTION OR TREATMENT OF MUSCLE DISEASE

(71) Applicant: Daewoong Pharmaceutical Co., Ltd., Gyeonggi-do (KR)

(72) Inventors: Jung Min KOH, Seoul (KR); Sung Sub KIM, Daejeon (KR); Kyong Hoon AHN, Seoul (KR)

(73) Assignee: Daewoong Pharmaceutical Co., Ltd., Gyeonggi-do (KR)

(21) Appl. No.: 17/434,388

(22) PCT Filed: Feb. 27, 2020
(86) PCT No.: PCT/KR2020/002809

§ 371 Date: Aug. 26, 2021

(30) Foreign Application Priority Data
Feb. 27, 2019 (KR) 10/2019-0023375

(51) Int. Cl. A61K 38/38; A61K 47/64; A61P 21/00; A61K 9/00

(52) U.S. Cl. CPC A61K 38/38; A61K 9/0019; A61P 21/00; A61K 47/643

(57) ABSTRACT

The present invention relates to a composition comprising albumin-bound Slit3 LRRD2 for prevention or treatment of a muscle disease, and more particularly provides a fusion protein comprising albumin-bound Slit3 LRRD2, a nucleic acid molecule encoding the fusion protein, a recombinant vector comprising the nucleic acid molecule, a transformant comprising the recombinant vector, a method for preparing a fusion protein using the transformant, a composition comprising the fusion protein for prevention or treatment of a muscle disease, and a composition comprising the fusion protein for improving the in vivo half-life of LRRD2 of the Slit 3 protein.

### TECHNICAL FIELD

The present invention relates to a composition comprising albumin-bound Slit3 LRRD2 for prevention or treatment of a muscle disease, and more particularly provides a fusion protein comprising albumin-bound Slit3 LRRD2, a nucleic acid molecule encoding the fusion protein, a recombinant vector comprising the nucleic acid molecule, a transformant comprising the recombinant vector, a method for preparing a fusion protein using the transformant, a composition comprising the fusion protein for prevention or treatment of a muscle disease, and a composition comprising the fusion protein for improving the in vivo half-life of LRRD2 of the Slit 3 protein.

### BACKGROUND ART

Slit proteins are well-known proteins that regulate the movement of neurons and axons during the developmental process of the nervous system. It is known that a Slit protein can act with a Robo receptor to regulate physiological activity, and serves as a factor that regulates various intracellular processes in various tissues such as heart, lung, kidney, and breast tissues, and as it has been recently reported that Slit proteins play an important role in the regulation of growth, adhesion ability, and migration ability of cells, it was reported that Slit proteins can participate in the migration in the differentiation of cells and the occurrence and metastasis of cancer. Specifically, it was reported that Slit proteins and Robo proteins are expressed in the embryonic development stage of a vertebrate, and the expression of Slit3, Robo1 and Robo2 proteins is increased in the muscle tissues. The report mentioned that the expression of the Slit3 protein was increased in the myoblasts of hind leg muscle tissues of an embryo, but the protein might only participate in the migration ability and might not be associated with the differentiation of myoblasts.

The present inventors have revealed that LRRD2 of the Slit3 protein can be used for prevention or treatment of sarcopenia by promoting the differentiation of myoblasts to induce an increase in muscle mass (Korean Patent Application Laid-Open No. 10-2017-0138920). Since LRRD2 needs to be administered as an injection, patients need to visit the hospital, but LRRD2 has a very short in vivo half-life, so its administration cycle should be shortened in order to exhibit the medicinal effects thereof, and it is expected that a problem in that its efficacy is reduced due to the associated excessive use of the drug occurs.

Thus, the present inventors have developed an HSA-Slit3 LRRD2 fusion protein with improved efficacy by increasing the in vivo half-life of LRRD2, thereby completing the present invention.

### DISCLOSURE

### Technical Problem

An object of the present invention is to provide a fusion protein for improving the in vivo half-life of LRRD2 of the Slit3 protein.

Another object of the present invention is to provide a nucleic acid molecule encoding the above-described fusion protein, a recombinant vector comprising the nucleic acid molecule, and a transformant comprising the recombinant vector.

Still another object of the present invention is to provide a method for preparing the above-described fusion protein.

Yet another object of the present invention is to provide a composition in which LRRD2 of the Slit3 protein has improved preventive or treatment efficacy for a muscle disease.

Yet another object of the present invention is to provide a composition in which LRRD2 of the Slit3 protein has an enhanced in vivo half-life.

### Technical Solution

To achieve the above-described objects, the present invention provides a fusion protein in which albumin is bound to LRRD2 of the Slit3 protein.

According to a preferred exemplary embodiment of the present invention, the albumin may be human serum albumin.

According to another preferred exemplary embodiment of the present invention, in the fusion protein, the human serum albumin may be bound to the N-terminus of LRRD2 of the Slit3 protein.

According to still another preferred exemplary embodiment of the present invention, the human serum albumin may include an amino acid sequence of SEQ ID NO: 2.

According to yet another preferred exemplary embodiment of the present invention, the LRRD2 of the Slit3 protein may include an amino acid sequence of SEQ ID NO: 3.

According to yet another preferred exemplary embodiment of the present invention, a linker may be further included between the albumin and the LRRD2 of the Slit3 protein.

According to yet another preferred exemplary embodiment of the present invention, the linker may be (GGGGS)n (SEQ ID NO: 5), wherein n may be an integer from 1 to 10.

The present invention also provides a nucleic acid molecule encoding the above-described fusion protein.

The present invention also provides a recombinant vector comprising the above-described nucleic acid molecule and a transformant comprising the same.

The present invention also provides a method for preparing a fusion protein, the method comprising culturing the above-described transformant.

The present invention also provides a pharmaceutical composition comprising the above-described fusion protein for prevention or treatment of a muscle disease.

According to yet another preferred exemplary embodiment of the present invention, the pharmaceutical composition may be administered as an injection.

According to yet another preferred exemplary embodiment of the present invention, the muscle disease may be any one or more selected from the group consisting of atony, muscular atrophy, muscular dystrophy, cachexia, and sarcopenia.

The present invention also provides a composition comprising albumin-bound LRRD2 of the Slit3 protein for improving the in vivo half-life of LRRD2 of the Slit3 protein.

### Advantageous Effects

Since the albumin-bound LRRD2 of the Slit3 protein exhibits the same cytological efficacy as albumin-unbound LRRD2 of the Slit3 protein and has a significantly increased in vivo half-life compared to albumin-unbound LRRD2 of the Slit3 protein, a bone-related disease can be more effectively prevented or treated.

### DESCRIPTION OF DRAWINGS

FIG. 1 illustrates a composition of a fusion protein in which albumin of the present invention is bound to the N-terminus of Slit3 LRRD2 and an amino acid sequence thereof.

FIG. 2 illustrates the results of performing SDS-PAGE after isolating and purifying an SP cystatin S-HSA-Slit3LRRD2 fusion protein.

FIG. 3 graphically illustrates the receptor binding ability of various forms of HSA-Slit3 LRRD2 fusion proteins.

FIG. 4 illustrates the plasma concentration-time profiles of Slit3 LRRD2 after IV administration of Slit3 LRRD2 (●, “Slit3”) and HSA-Slit3 LRRD2 (▪, “HSA-Slit3”) to fasted male ICR mice.

### MODES OF THE INVENTION

As described above, LRRD2 of the Slit3 protein may be used for prevention or treatment of sarcopenia by promoting the differentiation of myoblasts to induce an increase in muscle mass, but LRRD2 has a very short in vivo half-life, so its administration cycle should be shortened in order to exhibit the medicinal effects thereof, and it is expected that a problem in that its efficacy is reduced due to the associated excessive use of the drug occurs

Thus, the present inventors have sought a solution to the above-described problem by enhancing the in vivo half-life of LRRD2 to develop an HSA-Slit3 LRRD2 fusion protein with improved efficacy. Since the albumin-bound LRRD2 of the Slit3 protein exhibits the same cytological efficacy as albumin-unbound LRRD2 of the Slit3 protein and has a significantly increased in vivo half-life compared to albumin-unbound LRRD2 of the Slit3 protein, a muscle-related disease can be more effectively prevented or treated.

Hereinafter, the present invention will be described in more detail.

The present invention provides a fusion protein in which albumin is bound to LRRD2 of the Slit3 protein.

In the fusion protein of the present invention, the “LRRD2 of the Slit3 protein” refers to a second leucine-rich repeat domain (LRRD2) in the Slit3 protein. Through previous studies, the present inventors confirmed that the Slit3 protein or LRRD2 in this protein binds to the Robo1 or Robo2 receptor to release the β-catenin binding to the M-cadherin of myoblasts via the Slit-Robo system, thereby promoting the formation of muscles by activating the β-catenin and increasing the expression of myogenin to induce the differentiation of myoblasts. As used herein, the term “Slit3 LRRD2” refers to “LRRD2 of the Slit3 protein” and may be used interchangeably.

In the fusion protein of the present invention, the albumin may be human serum albumin, rhesus serum albumin (RhSA), cynomolgus monkey serum albumin (CySA), or murine serum albumin (MuSA), and preferably human serum albumin. The Slit3 LRRD2 has an in vivo half-life of Slit3 LRRD2 in the presence of human serum albumin that is at least 10-fold longer than that of Slit3 LRRD2 in the absence of human serum albumin. In a specific exemplary embodiment of the present invention, the serum half-life of Slit3 LRRD2 in the presence of human serum albumin is 14-fold longer than that of Slit3 LRRD2 in the absence of human serum albumin.

The fusion protein according to the present invention may be bound in the order of the human serum albumin and Slit3 LRRD2, or vice versa. Preferably, the human serum albumin and Slit3 LRRD2 are bound in this order. For example, when the human serum albumin binds to the N-terminus of Slit3 LRRD2, Slit3 LRRD2 has the best in vivo half-life and therapeutic efficacy for muscle-related diseases, and when the human serum albumin binds to the C-terminus thereof, it is possible to exhibit an effective efficacy even though there may be a difference in degree.

In the fusion composition of the present invention, as the human serum albumin, a full-length amino acid sequence consisting of 609 amino acids or a fragment comprising a partial amino acid sequence thereof may be used. The full-length amino acid sequence of the human serum albumin is disclosed in the NCBI GenBank: AAA98797.1, and in an exemplary embodiment of the present invention, the form of a fragment consisting of the 25th to 609th amino acids (585 amino acids) from the full-length human serum albumin consisting of 609 amino acids was used. In the fusion protein of the present invention, the human serum albumin consists of the following SEQ ID NO: 2:

(SEQ ID NO: 2)
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL.

In the fusion protein of the present invention, the Slit3 LRRD2 is human-derived, and a full-length amino acid sequence of LRRD2 in the Slit3 protein consisting of 1523 amino acids or a fragment comprising a partial amino acid sequence thereof may be used. The full-length amino acid sequence of the Slit3 protein is disclosed in the NCBI GenBank: AAQ89243.1, and in an exemplary embodiment of the present invention, as Slit3 LRRD2, the form of a fragment consisting of the 278th to 486th amino acids (209 amino acids) from the full-length Slit3 protein consisting of 1523 amino acids was used. In the fusion protein of the present invention, Slit3 LRRD2 consists of an amino acid sequence of the following SEQ ID NO: 3.

(SEQ ID NO: 3)
ISCPSPCTCSNNIVDCRGKGLMEIPANLPEGIVEIRLEQNSIKAIPAGAF
TQYKKLKRIDISKNQISDIAPDAFQGLKSLTSLVLYGNKITEIAKGLFDG
LVSLQLLLLNANKINCLRVNTFQDLQNLNLLSLYDNKLQTISKGLFAPLQ
SIQTLHLAQNPFVCDCHLKWLADYLQDNPIETSGARCSSPRRLANKRISQ
IKSKKFRCS.

In the fusion protein of the present invention, “Slit3 LRRD2” may include a functional equivalent of the amino acid sequence of SEQ ID NO: 3.

The “functional equivalent” has a sequence homology of at least 70% or more, preferably 80% or more, more preferably 90% or more, and even more preferably 95% or more with the amino acid sequences of SEQ ID NOS: 1 to 4 of the present invention by the addition, substitution, or deletion of amino acids of a protein or peptide, and refers to a protein or peptide exhibiting physiological activity substantially equivalent to that of a protein or peptide consisting of amino acid sequences of SEQ ID NOS: 1 to 4.

Specifically, for the fusion protein, not only a protein or peptide having a wild-type amino acid sequence thereof, but also an amino acid sequence variant thereof may also be included in the scope of the present invention. The amino acid sequence variant refers to a protein or peptide having a sequence different from a wild-type amino acid sequence of Slit3 LRRD2 by deletion, insertion, non-conservative or conservative substitution of one or more amino acid residues, or a combination thereof.

Amino acid exchanges possible in proteins and peptides that do not entirely change the activities of the molecules are known in the art (H. Neurath, R. L. Hill, The Proteins, Academic Press, New York, 1979). The most typically occurring exchanges are exchanges between amino acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly. In some cases, amino acids may also be modified by phosphorylation, sulfation, acetylation, glycosylation, methylation, farnesylation, or the like.

The Slit3 LRRD2 of the present invention, or variants thereof can be extracted from nature or synthesized (Merrifield, J. Amer. Chem. Soc. 85:2149-2156, 1963) or prepared by a gene recombinant method based on a DNA sequence (Sambrook et al., Molecular Cloning, Cold Spring Harbour Laboratory Press, New York, USA, 2d Ed., 1989).

The Slit3 LRRD2 of the present invention may be subjected not only to albumin fusion, but also to fusion or PEGylation of an Fc protein of IgG, and the like in order to enhance the in vivo half-life thereof.

In the fusion protein of the present invention, a linker may be further included between albumin and LRRD2 of the Slit3 protein. A preferred linker type may be (GGGS)n (SEQ ID NO: 5), wherein n may be an integer from 1 to 10, and preferably n may be an integer from 1 to 5.

The present invention also provides a nucleic acid molecule encoding a fusion protein in the form of human serum albumin being linked to the N-terminus of Slit3 LRRD2, and additionally, cystatin S may be linked to the N-terminus of the human serum albumin.

In the nucleic acid molecule of the present invention, cystatin S is a signal peptide, a base sequence encoding the cystatin S is linked to the 5′ terminus of a base sequence encoding a human serum albumin, and a base sequence encoding the human serum albumin may be linked to the 5′ terminus of a base sequence encoding Slit3 LRRD2 to produce cystatin S-HSA-Slit3 LRRD.

In the nucleic acid molecule of the present invention, the cystatin S is human-derived, and may be used as a base sequence encoding a full-length amino acid sequence consisting of 141 amino acids or a partial amino acid sequence thereof. The full-length amino acid sequence of cystatin S is disclosed in the NCBI GenBank: EAX10135.1, and in an exemplary embodiment of the present invention, in a full-length cystatin S consisting of 141 amino acids, a base sequence encoding a fragment consisting of the 1st to 20th amino acids was used. In the nucleic acid molecule of the present invention, cystatin S is a base sequence encoding an amino acid sequence of the following SEQ ID NO: 1.

(SEQ ID NO: 1)
MARPLCTLLLLMATLAGALA.

The present invention also provides a recombinant vector comprising a base sequence encoding the fusion protein and a promoter functionally linked to the base sequence.

The term “functionally linked” means being functionally linked between a nucleic acid expression regulatory sequence (an array of a promoter, a signal sequence, or a transcriptional regulatory factor binding site) and a secondary base sequence, and the expression regulatory sequence affects the transcription and/or translation of nucleic acids corresponding to the secondary sequence.

The vector system of the present invention may be prepared by a method well-known in the art as described in the literature [Sambrook et al., Molecular Cloning, A Laboratory Manual, Cold Spring Harbor Laboratory Press (2001)].

In general, the vector may be prepared for cloning or expression purposes. Further, the vector may be prepared for use in eukaryotic or prokaryotic host cells. For example, when a vector is prepared for expression in prokaryotic cells, the vector includes a strong promoter for initiating transcription (for example, a pLk promoter, a trp promoter, a lac promoter, a tac promoter and a T7 promoter), a ribosome binding site or a sequence for initiating translation and stopping transcription/translation. In particular, when E. coli is used as a host cell, for biosynthesis of tryptophan in E. coli, a promoter and an operator in an operon (Yanofsky, C., J. Bacteriol., 158: 1018-1024 (1984)) and a left directional promoter of phage λ (a pLλ promoter, Herskowitz, I. and Hagen, D., Ann. Rev. Genet., 14: 399-445 (1980)) may be used as regulatory sequences. When Bacillus is used as a host cell, a promoter for a gene encoding a toxin protein of Bacillus thuringiensis (Appl. Environ. Microbiol. 64: 3932-3938 (1998); and Mol. Gen. Genet. 250: 734-741 (1996)) or another operable promoter in Bacillus may be used as a regulatory sequence.

Numerous typical vectors available for prokaryotic cells are well known to those skilled in the art, and selection of a suitable vector is a matter of choice. A typical vector used in the present invention includes pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, λgt ⋅4 λB, λ-charon, λΔz1 and M13 and is not necessarily limited thereto.

For example, when a vector is prepared for a eukaryotic host cell, among them, a promoter derived from the genome of an animal cell or a mammalian cell (for example, a metallothionein promoter) or a mammalian virus (for example, an adenovirus late promoter; a vaccinia 7.5 K promoter, an SV40 promoter, a cytomegalovirus promoter and a tk promoter of HSV) may be used. The vector generally includes a poly adenylation site of a transcript. Examples of a commercially available virus-based vector include pcDNA 3 (Invitrogen; including a cytomegalovirus promoter and a polyadenylation signal), pSI (Promega; including an SV 40 promoter and a polyadenylation signal), pCI (Promega; including a cytomegalovirus promoter and a polyadenylation signal), and pREP7 (Invitrogen; including a RSV promoter and an SV 40 polyadenylation signal).

When a vector is prepared for yeast, the promoters of the genes for phosphoglycerate kinase, glyceraldehyde-3-phosphate dehydrogenase, lactase, enolase and alcohol dehydrolase may be used as regulatory sequences.

When an expression vector is prepared for plant cells, various plant functional promoters known in the art may be used, and include a cauliflower mosaic virus (CaMV) 35S promoter, the pigwarm mosaic virus 35S promoter, a sugarcane bacilliform virus promoter, a Comerina yellow mottle virus promoter, alight-inducible promoter from subunit of ribulose-1,5-bis-phosphate carboxylase (ssRUBISCO), a rice cytosolic triosephosphate isomerase (TPI) promoter, an adenine phosphoribosyltransferase (APRT) promoter from Arabidopsis, a rice actin 1 gene promoter and mannopine synthase and octopine synthase promoters.

In addition, the recombinant vector of the present invention includes a base sequence capable of easily separating an expressed fusion protein, and includes, but is not limited to, glutathione S-transferase (Pharmacia, USA), a maltose binding protein (NEB, USA), FLAG (IBI, USA) and 6×His (hexahistidine; Qiagen, USA). Fusion proteins expressed by such an additional sequence may be separated by affinity chromatography in a rapid and convenient manner.

In a specific exemplary embodiment of the present invention, a FLAG sequence was used, and a FLAG protein includes an amino acid sequence of SEQ ID NO: 4. The amino acid sequence of SEQ ID NO: 4 is as follows:

(SEQ ID NO: 4)
DYKDDDDK.

According to another exemplary embodiment of the present invention, the fusion protein is separated by affinity chromatography. For example, in the case of glutathione S-transferase, an elution buffer including glutathione is used, and when 6×His is used to separate a foreign protein, a Ni-NTA His-binding resin (Novagen, USA) is used.

The expression vector of the present invention preferably includes one or more markers capable of selecting a transformed host, and examples thereof include genes resistant to antibiotics such as ampicillin, gentamicin, chloramphenicol, streptomycin, kanamycin, neomycin, geneticin and tetracycline, URA3 genes, and genes having resistance to other toxic compounds such as metal ions.

The present invention also provides a transformant comprising the recombinant vector described above.

A host useful for preparing a transformant is well-known in the art. For example, it is possible to use a prokaryotic host, E. coli JM109, E. coli BL21, E. coli RR1, E. coli LE392, E. coli B, E. coli X 1776, E. coli W3110, Bacillus subtilis, Bacillus thuringiensis, Salmonella typhimurium, Serratia marcescens and various Pseudomonas species, Corynebacterium and Streptomyces.

In eukaryotic cells, yeast (Saccharomyces cerevisiae), insect cells, human cells (for example, CHO, W138, BHK, COS-7, 293, HepG2, 3T3, RIN and MDCK cell lines) and plant somatic cells may be used.

Transformation of host cells may be performed by a number of methods known in the art. For example, when prokaryotic cells are used as host cells, a CaCl₂) method, a Hanson method (Cohen, S. N. et al., Proc. Natl. Acac. Sci. USA, 9:2110-2114(1973); and Hanahan, D., J. Mol. Biol., 166:557-580(1983)) and electrophoresis may be used for transformation. Further, when eukaryotic cells are used as host cells, microinjection, calcium phosphate precipitation, electric shock, liposome-mediated phenotypic infection, DEAE-dextran treatment, and particle bombardment may be used for transformation. In addition, when plant cells are used as host cells, Agrobacterium-mediated transformation is the most preferable method, the reason for which is enabling a bypass required for redifferentiation of adjacent plants from protoplasts.

The present invention also provides a method for producing the HSA-Slit3 LRRD2 fusion protein. The method includes (a) culturing the transformant described above under conditions for expression; and (b) obtaining a produced fusion protein.

A transformant for preparing the fusion protein of the present invention may be cultured using a suitable medium and culture conditions known in the art. These culturing processes may be easily adjusted and used by those skilled in the art according to the selected strain. Cell culture is classified into suspension culture and adherent culture depending on the cell growth method, and into a batch culture type, a fed-batch type and a continuous culture type depending on the culture method. Media used for culturing need to appropriately meet the requirements of a particular strain.

A medium used for culturing animal cells contains various carbon sources, nitrogen sources, and trace element components. Examples of a carbon source which may be used include carbohydrates such as glucose, sucrose, lactose, fructose, maltose, starch and cellulose, fats such as soybean oil, sunflower oil, hemp oil and coconut oil, fatty acids such as palmitic acid, stearic acid and linoleic acid, alcohols such as glycerol and ethanol, and organic acids such as acetic acid. These carbon sources may be used either alone or in combination. Examples of a nitrogen source which may be used include organic nitrogen sources such as peptone, yeast extract, meat juice, malt extract, corn steep liquid (CSL) and soybean meal and inorganic nitrogen sources such as urea, ammonium sulfate, ammonium chloride, ammonium phosphate, ammonium carbonate and ammonium nitrate. These nitrogen sources may be used either alone or in combination. The medium may include potassium dihydrogen phosphate, dipotassium hydrogen phosphate and the corresponding sodium-containing salt as a phosphorus source. Furthermore, the medium may include a metal salt such as magnesium sulfate or iron sulfate. In addition to the above, amino acids, vitamins, an appropriate precursor, and the like may be included.

During the culturing, the pH of a culture may be adjusted by adding a compound such as ammonium hydroxide, potassium hydroxide, ammonia, phosphoric acid and sulfuric acid to the cultured product. Further, the formation of bubbles may be suppressed using a defoaming agent such as fatty acid polyglycol ester. In addition, in order to maintain the aerobic state of the culture, oxygen or an oxygen-containing gas (for example, air) is injected into the culture. The temperature of the culture is usually 20° C. to 45° C., preferably 25° C. to 40° C.

In the method for producing the HSA-Slit3 LRRD2 fusion protein of the present invention, the obtaining of the produced fusion protein in step (b) may be performed in order to obtain a fusion protein in a separated form. For example, when a fusion protein is expressed by a large volume of transformed bacteria, the fusion protein is generally expressed after promoter induction, but expression continues, and the protein forms an insoluble precipitate (that is, an inclusion body). There are several suitable protocols for the separation of the inclusion body. The fused protein that formed the inclusion body may be reformed through dilution or dialysis using a suitable buffer. And then, the fusion protein may be purified using a basic method known in the art including solubility fragmentation by the use of ammonium sulfate, size differential filtration (ultrafiltration) and column chromatography (depending on size, net surface charge, hydrophobicity or affinity).

The fusion protein of the present invention may be expressed in plants. Plant cells may be transformed by a typical method known in the art, and may be transformed by electric shock, particle bombardment and Agrobacterium-induced transformation. Among them, Agrobacterium-induced transformation is most preferred. Agrobacterium-induced transformation may generally be performed using leaf slices and other tissues such as cotyledon and hypocotyl.

Transformed cells may be selected by exposing a transformed culture to a selected preparation such as a metabolic inhibitor, an antibiotic and an herbicide. When cells are transformed and stably express a marker gene having resistance to a selected gene, the cells grow and differentiate during the culturing. For example, a marker includes, but is not limited to, a glycophospate resistant gene and a neomycin phosphotransferase (nptII) system. The development or redifferentiation of plants from plant protoplasts or various foreign materials is well known in the art. The resulting transformed root shoots are seeded in a suitable plant growth medium. Development or redifferentiation of plants including foreign genes induced by Agrobacterium may be performed by methods known in the art.

The present invention provides a pharmaceutical composition comprising albumin-bound LRRD2 of the Slit3 protein for prevention or treatment of a muscle disease.

The pharmaceutical composition of the present invention may be in the form of various oral or parenteral formulations. When the pharmaceutical composition is formulated, the composition may be prepared by using a buffer (for example, a saline solution or PBS), an antioxidant, a bacteriostatic agent, a chelating agent (for example, EDTA or glutathione), a filler, an extender, a binder, an adjuvant (for example, aluminum hydroxide), a suspension agent, a thickener, a wetting agent, a disintegrant, or a surfactant, a diluent or an excipient.

Examples of a solid preparation for oral administration include a tablet, a pill, a powder, granules, a capsule, and the like, and the solid preparation is prepared by mixing one or more compounds with one or more excipients, for example, starch (including corn starch, wheat starch, rice starch, potato starch, and the like), calcium carbonate, sucrose, lactose, dextrose, sorbitol, mannitol, xylitol, erythritol maltitol, cellulose, methyl cellulose, sodium carboxymethylcellulose and hydroxypropymethyl-cellulose, gelatin, or the like. For example, a tablet or a sugar tablet may be obtained by blending an active ingredient with a solid excipient, pulverizing the resulting blend, adding a suitable auxiliary agent thereto, and then processing the resulting mixture into a granular mixture.

Further, in addition to simple excipients, lubricants such as magnesium stearate and talc are also used. A liquid preparation for oral administration corresponds to a suspension agent, a liquid for internal use, an emulsion, a syrup, and the like, and the liquid preparation may include, in addition to water and liquid paraffin which are simple commonly used diluents, various excipients, for example, a wetting agent, a sweetener, an odorant, a preservative, and the like. In addition, in some cases, cross-linked polyvinyl pyrrolidone, agar, alginic acid, sodium alginate, or the like may be added as a disintegrant, and an anti-coagulant, a lubricant, a wetting agent, a flavoring agent, an emulsifier, an antiseptic, and the like may be additionally added.

Examples of a preparation for parenteral administration include an aqueous sterile solution, a non-aqueous solvent, a suspension solvent, an emulsion, a freeze-dried preparation, a suppository, or the like. As the non-aqueous solvent and the suspension solvent, it is possible to use propylene glycol, polyethylene glycol, a vegetable oil such as olive oil, an injectable ester such as ethyl oleate, and the like. As a base of the suppository, it is possible to use Witepsol, Macrogol, Tween 61, cacao butter, laurin fat, glycerol, gelatin, and the like.

The pharmaceutical composition of the present invention may be administered orally or parenterally, and, when administered parenterally, may be formulated in the form of a preparation for external application to the skin; an injection administered intraperitoneally, rectally, intravenously, muscularly, subcutaneously, or intracerebroventricularly, or via cervical intrathecal injection; a percutaneous administration agent; or a nasal inhaler according to a method known in the art.

The injection must be sterilized and protected from contamination of microorganisms such as bacteria and fungi. Examples of a suitable carrier for the injection may be, but are not limited to, a solvent or a dispersion medium including water, ethanol, polyols (for example, glycerol, propylene glycol, liquid polyethylene glycol, and the like), mixtures thereof, and/or vegetable oils. More preferably, as a suitable carrier, it is possible to use an isotonic solution such as Hank's solution, Ringer's solution, triethanolamine-containing phosphate buffered saline (PBS) or sterile water for injection, 10% ethanol, 40% propylene glycol, and 5% dextrose, and the like. To protect the injection from microbial contamination, various antimicrobial agents and antifungal agents such as a paraben, chlorobutanol, phenol, sorbic acid, and thimerosal may be additionally included. Furthermore, in most cases, the injection may additionally include an isotonic agent such as sugar or sodium chloride.

Examples of the percutaneous administration agent include a form such as an ointment, a cream, a lotion, a gel, a solution for external use, a paste, a liniment, and an aerosol. The transdermal administration as described above means that an effective amount of an active ingredient contained in a pharmaceutical composition is delivered into the skin via local administration thereof to the skin.

In the case of a preparation for inhalation, the fusion protein used according to the present invention may be conveniently delivered in the form of an aerosol spray from a pressurized pack or a nebulizer by using a suitable propellant, for example, dichlorofluoromethane, trichlorofluoromethane, dichlorotetrafluoroethane, carbon dioxide, or other suitable gases. In the case of the pressurized aerosol, a dosage unit may be determined by providing a valve for transferring a metered amount. For example, a gelatin capsule and a cartridge for use in an inhaler or insufflator may be formulated so as to contain a powder mixture of a compound and a suitable powder base such as lactose or starch. Formulations for parenteral administration are described in the document, which is a guidebook generally known in all pharmaceutical chemistry fields (Remington's Pharmaceutical Science, 15th Edition, 1975. Mack Publishing Company, Easton, Pa. 18042, Chapter 87: Blaug, Seymour).

The pharmaceutical composition of the present invention is administered in a pharmaceutically effective amount. The term “pharmaceutically effective amount” as used herein refers to an amount sufficient to treat diseases at a reasonable benefit/risk ratio applicable to medical treatment, and an effective dosage level may be determined according to factors including type of diseases of patients, the severity of disease, the activity of drugs, sensitivity to drugs, administration time, administration routes, excretion rate, treatment periods, and simultaneously used drugs, and other factors well known in the medical field. The pharmaceutical composition of the present invention may be administered as an individual therapeutic agent or in combination with other therapeutic agents, may be administered sequentially or simultaneously with therapeutic agents in the related art, and may be administered in a single dose or multiple doses. That is, the total effective amount of the composition of the present invention may be administered to a patient in a single dose or may be administered by a fractionated treatment protocol, in which multiple doses are administered over a long period of time. It is important to administer the composition in a minimum amount that can obtain the maximum effect without any side effects, in consideration of all the aforementioned factors, and this amount may be easily determined by those skilled in the art.

A dosage of the pharmaceutical composition of the present invention varies according to body weight, age, gender, and health status of a patient, age of a patient, diet, administration time, administration method, excretion rate, and the severity of a disease. A daily dosage thereof may be administered parenterally in an amount of preferably 0.01 to 50 mg, and more preferably 0.1 mg to 30 mg per 1 kg of body weight a day based on HSA-Slit3 LRRD2, and a daily dosage thereof may be administered orally in a single dose or multiple doses in an amount of preferably 0.01 to 100 mg, and more preferably 0.01 to 10 mg per 1 kg of body weight a day based on the HSA-Slit3 LRRD2 of the present invention. However, since the effective amount may be increased or decreased depending on the administration route, the severity of obesity, gender, body weight, age, and the like, the dosage is not intended to limit the scope of the present invention in any way.

The pharmaceutical composition of the present invention may be used either alone or in combination with surgery, radiation therapy, hormone therapy, chemotherapy, and methods using a biological response modifier.

The pharmaceutical composition of the present invention may also be provided as a formulation for external application. When the pharmaceutical composition for preventing and treating a muscle disease according to the present invention is used as a preparation for external application to the skin, the pharmaceutical composition may additionally contain auxiliary agents typically used in the dermatology field, such as any other ingredients typically used in the preparation for external application to the skin, such as a fatty substance, an organic solvent, a solubilizing agent, a thickener and a gelling agent, a softener, an antioxidant, a suspending agent, a stabilizer, a foaming agent, an odorant, a surfactant, water, an ionic emulsifier, a non-ionic emulsifier, a filler, a metal ion blocking agent, a chelating agent, a preservative, a vitamin, a blocking agent, a wetting agent, an essential oil, a dye, a pigment, a hydrophilic active agent, a lipophilic active agent, or a lipid vesicle. In addition, the ingredients may be introduced in an amount generally used in the dermatology field.

When the pharmaceutical composition for preventing and treating a muscle disease according to the present invention is provided as a preparation for external application to the skin, the pharmaceutical composition may be in the form of a formulation such as an ointment, a patch, a gel, a cream, and an aerosol, but is not limited thereto.

It is preferred that the muscle disease of the present invention is a muscle disease caused by muscular function deterioration, muscle wasting, or muscle degeneration and is a disease reported in the art, and specifically, it is more preferred that the muscle disease of the present invention is one or more selected from the group consisting of atony, muscular atrophy, muscular dystrophy, muscle degeneration, myasthenia gravis, cachexia, and sarcopenia, but the muscle disease is not limited thereto.

The muscle wasting or degeneration occurs for reasons such as congenital factors, acquired factors, and aging, and the muscle wasting is characterized by a gradual loss of muscle mass, and weakening and degeneration of a muscle, particularly, a skeletal muscle or a voluntary muscle and a cardiac muscle.

More specifically, the muscle comprehensively refers to a sinew, a muscle, and a tendon, and the muscular function or muscle function means an ability to exert a force by contraction of the muscle, and includes: muscular strength in which the muscle can exert the maximum contraction force in order to withstand the resistance; muscular endurance strength which is an ability to exhibit how long or how many times the muscle can repeat the contraction and relaxation to a given weight; and quickness which is an ability to exert a strong force within a short period of time. The muscular function is proportional to the muscle mass, and the term “improvement of muscular function” refers to the improvement of the muscular function in a more positive direction.

The present invention also provides a health functional food composition comprising albumin-bound LRRD2 of the Slit3 protein for prevention or alleviation of a muscle disease. Since the composition of an active ingredient included in the health functional food composition of the present invention and effects thereof are the same as those for the above-described pharmaceutical composition, the description thereof will be omitted.

The health functional food composition according to the present invention can be prepared in various forms by typical methods known in the art. A general food can be prepared by adding the HSA-Slit3 LRRD2 fusion protein of the present invention to, without being limited to, a beverage (including an alcoholic beverage), fruit and a processed food thereof (for example: canned fruit, bottled food, jam, marmalade, and the like), fish, meat and processed food thereof (for example: ham, sausage, corned beef, and the like), bread and noodles (for example: thick wheat noodles, buckwheat noodles, instant noodles, spaghetti, macaroni, and the like), fruit juice, various drinks, cookies, wheat-gluten, dairy products (for example: butter, cheese, and the like), edible vegetable oils, margarine, vegetable protein, retort foods, frozen food and various seasonings (for example: soybean paste, soy sauce, sauce, and the like), and the like. In addition, a nutritional supplement can be prepared by adding the HSA-Slit3 LRRD2 fusion protein of the present invention to, without being limited to, a capsule, a tablet, a pill, and the like. Furthermore, for a health functional food, for example, the HSA-Slit3 LRRD2 fusion protein of the present invention itself is prepared in the form of, without being limited to, tea, juice, and drinks and can be taken by being processed into a liquid, granules, a capsule, and a powder so as to be able to be drunk (health beverage). Further, the HSA-Slit3 LRRD2 fusion protein of the present invention can be used and prepared in the form of a powder or a concentrated liquid so as to be used in the form of a food additive. In addition, the food functional composition of the present invention can be prepared in the form of a composition by mixing the HSA-Slit3 LRRD2 fusion protein of the present invention with an active ingredient known to have effects of preventing a muscle disease and improving muscular function.

When the HSA-Slit3 LRRD2 fusion protein of the present invention is used as a health beverage, the health beverage composition can contain various flavoring agents or natural carbohydrates, and the like as additional ingredients, such as atypical beverage. The above-described natural carbohydrates may be monosaccharides such as glucose and fructose; disaccharides such as maltose and sucrose; polysaccharides such as dextrin and cyclodextrin; and sugar alcohols such as xylitol, sorbitol, and erythritol. As a sweetener, it is possible to use a natural sweetener such as thaumatin and stevia extract; a synthetic sweetener such as saccharin and aspartame, and the like. The proportion of the natural carbohydrate is generally about 0.01 to 0.04 g, and preferably about 0.02 to 0.03 g per 100 mL of the composition of the present invention.

Furthermore, the HSA-Slit3 LRRD2 fusion protein of the present invention may be contained as an active ingredient of a food composition for preventing a muscle disease and improving muscular function, and the amount thereof is an amount effective to achieve an action for preventing a muscle disease and improving a muscular function and is not particularly limited, but is preferably 0.01 to 100 wt % based on the total weight of the entire composition. The health functional food composition of the present invention can be prepared by mixing the HSA-Slit3 LRRD2 fusion protein with other active ingredients known to have effects of preventing a muscle disease and improving a muscular function.

In addition to the aforementioned ingredients, the health functional food composition of the present invention may contain various nutrients, vitamins, electrolytes, flavoring agents, coloring agents, pectic acid, salts of pectic acid, alginic acid, salts of alginic acid, organic acids, protective colloidal thickeners, pH adjusting agents, stabilizers, preservatives, glycerin, alcohols, carbonate agents, and the like. In addition, the health food of the present invention may contain flesh for preparing natural fruit juice, fruit juice beverages, or vegetable beverages. These ingredients may be used either alone or in mixtures thereof. The proportion of these additives is not significantly important, but is generally selected within a range of 0.01 to 0.1 part by weight per 100 parts by weight of the composition of the present invention.

Further, the present invention provides a cosmetic composition comprising an HSA-Slit3 LRRD2 fusion protein for improving muscle function. Since the composition of an active ingredient included in the cosmetic composition of the present invention and effects thereof are the same as those for the above-described pharmaceutical composition, the description thereof will be omitted.

The cosmetic composition is not particularly limited, but may be used for external application to the skin.

The cosmetic composition of the present invention contains the HSA-Slit3 LRRD2 fusion protein as an active ingredient, and may be prepared in the form of a basic cosmetic composition (a lotion, a cream, an essence, a cleanser such as cleansing foam and cleansing water, a pack, and a body oil), a coloring cosmetic composition (a foundation, a lip-stick, a mascara, and a make-up base), a hair product composition (a shampoo, a rinse, a hair conditioner, and a hair gel), a soap, and the like with dermatologically acceptable excipients.

The excipient may include, for example, but not limited thereto, a skin softener, a skin infiltration enhancer, a coloring agent, an odorant, an emulsifier, a thickener, and a solvent. Further, it is possible to additionally include a fragrance, a pigment, a disinfectant, an antioxidant, a preservative, a moisturizer and the like, and to include a thickening agent, inorganic salts, a synthetic polymer material, and the like for improving physical properties. For example, when a cleanser and a soap are prepared by using the cosmetic composition of the present invention, the cleanser and the soap may be easily prepared by adding the HSA-Slit3 LRRD2 fusion protein to a typical cleanser and soap base. When cream is prepared, the cream may be prepared by adding the HSA-Slit3 LRRD2 fusion protein to a general oil-in-water (O/W) cream base. It is possible to further add a fragrance, a chelating agent, a pigment, an antioxidant, a preservative, and the like, and synthetic or natural materials such as proteins, minerals or vitamins for improving physical properties thereto.

The content of the HSA-Slit3 LRRD2 fusion protein contained in the cosmetic composition of the present invention is, but not limited to, preferably 0.001 to 10 wt %, and more preferably 0.01 to 5 wt %, based on the total weight of the entire composition. When the content is less than 0.001 wt %, desired effects cannot be expected, and when the content exceeds 10 wt %, it may be difficult to prepare the cosmetic composition of the present invention for reasons such as safety or formulation.

The present invention also provides a feed additive comprising albumin-bound LRRD2 of the Slit3 protein for improving muscle function. Since the composition of an active ingredient included in the feed additive of the present invention and effects thereof are the same as those for the above-described pharmaceutical composition, the description thereof will be omitted.

The feed additive of the present invention corresponds to a supplementary feed under the Control of Livestock and Fish Feed Act.

As used herein, the term “feed” may refer to any natural or artificial formula, one-meal, and the like, or a component of the one-meal for an animal to eat, ingest, and digest, or suitable for that.

The type of feed described above is not particularly limited, and a feed typically used in the art may be used. Non-limiting examples of the feed include vegetable feeds such as cereals, roots and fruits, food processing by-products, algae, fibers, pharmaceutical by-products, fats and oils, starches, gourds or grain by-products; and animal feeds such as proteins, inorganic substances, fats and oils, minerals, single cell proteins, animal plankton or foods. These feeds may be used alone or in mixtures of two or more thereof.

In addition, the feed additive may additionally contain a carrier that is acceptable to a unit animal. In the present invention, the feed additive may be used as it is, or a known carrier, stabilizer and the like may be added, various nutrients such as vitamins, amino acids, and minerals, antioxidants, other additives, and the like may be added, if necessary, and the shape thereof may be in a suitable state such as a powder, granules, a pellet, and a suspension. When the feed additive of the present invention is supplied, the feed additive may be supplied to a unit animal alone or in mixtures with the feed.

The present invention also provides a composition comprising albumin-bound LRRD2 of the Slit3 protein for improving the in vivo half-life of LRRD2 of the Slit3 protein.

Hereinafter, the present invention will be described in more detail through Examples. These Examples are only for exemplifying the present invention, and it should be obvious to a person with ordinary skill in the art that the scope of the present invention is not to be interpreted as being limited by these Examples.

The abbreviations used in the examples and meanings thereof are as shown in the following Table 1.

TABLE 1

| | |
|---|---|
| CL | Systemic plasma clearance |
| T_{1/2} | Terminal half-life |
| Vₛₛ | Steady state volume of distribution |
| IV | Intravenous |
| PO | Per oral |
| Cmₐₓ | Maximum plasma concentration observed |
| Tₘₐₓ | Time to Cmax |
| AUC_{0-∞} | Total area under the plasma concentration |
| | time curve from zero to infinity |
| AUC₀₋ₜ | Area under the plasma concentration time curve |
| | from zero to the last quantifiable time point |
| MRT | Mean residence time |
| BA | Estimated bioavailability |
| BQL | Below Quantification Level |

### Example 1

Preparation of HSA-Slit3 LRRD2 Fusion Protein

Expression was performed by transforming Expi293F suspension cells with 1.6 mg/ml PC DNA3.1 vector SP cystatin S-HSA-Slit3 LRR D2-FLAG DNA. After cells were cultured to 4.5 to 5×10⁶ cells/ml in 125 ml of a 293F cell suspension and only the medium was replaced with a new medium, transfection was performed by reacting 400 μl of Expifectamine with 7.5 ml of (A Sample) at room temperature for 5 minutes, reacting 150 ug of DNA with 7.5 ml of Opti-mem (B Sample) at room temperature for 5 minutes, and then mixing A and B Samples to react A and B Samples at room temperature for 20 minutes. After 24 hours, cells were treated by mixing Enhancers 1 and 2, and then cultured for 7 days.

After cells were precipitated from the culture solution cultured for 7 days using a centrifuge at 4° C. and 800 rpm for 20 minutes, the supernatant was filtered with a 0.22 μm filter manufactured by Corning and used. As a resin, an anti-FLAG resin manufactured by Sigma was used. 1.2 ml of the resin was respectively used, and purification was performed at 1 ml/min at 4° C. A washing buffer using Tris glycine (TBS, pH 7.4) was flowed in an amount which is 20-fold higher than that of the resin. For elution, 200 μl of a FLAG peptide manufactured by Sigma-Aldrich and 9.8 ml of TBS were mixed and used, 8 pieces of 500 μl per fraction were obtained, protein fractions were collected, concentrated by changing the buffer to DPBS, and then the concentration was measured.

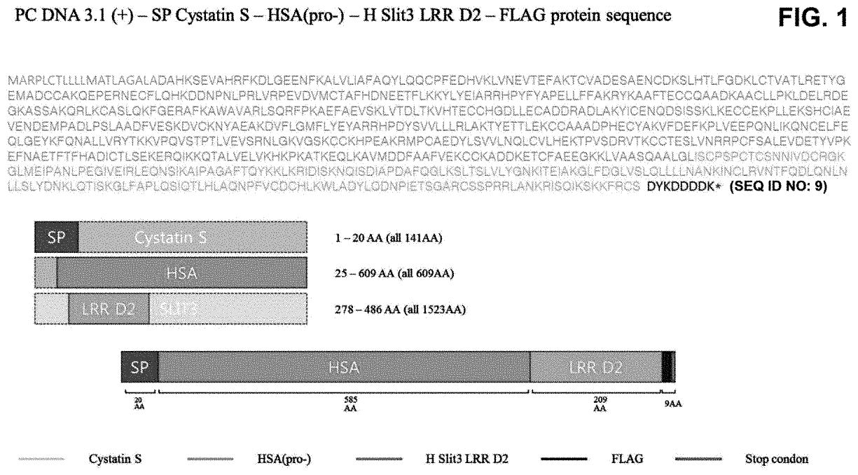

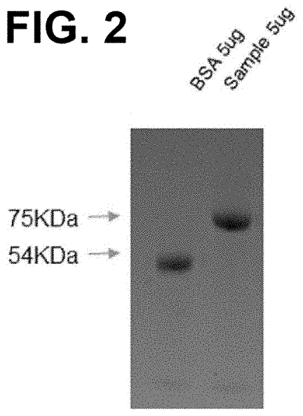

FIG. 2 illustrates the results of performing SDS-PAGE after isolating and purifying the fusion protein by the above process, confirming that the size of the fusion protein illustrated in FIG. 1, which was prepared in the present example, was 75 KDa.

### Example 2

Confirmation of Receptor Binding Ability of Various Forms of HSA-Slit3 LRRD2 Fusion Proteins

2-1. Preparation of Various Forms of HSA-Slit3 LRRD2 Fusion Proteins

Based on the preparation method of Example 1, 12 types of various HSA-Slit3 LRRD2 fusion proteins were prepared as shown in the following Table 2. As a linker, (GGGGS)₃ (SEQ ID NO: 6) was used.

TABLE 2

| Type of | Terminus | Presence or | | |
|---|---|---|---|---|
| fusion | bound to | absence of | Type of | |
| protein | HSA | linker | LRRD2 | Final form |
| LRRD2-1 | N terminus | None | Fragment | HSA-Fragment |
| | | | (68 a.a.) | LRRD2 |
| LRRD2-2 | N terminus | None | Intermediate | HSA-Intermediate |
| | | | (130 a.a) | LRRD2 |
| LRRD2-3 | N terminus | None | Full-length | HSA-Full-length |
| | | | (209 a.a) | LRRD2 |
| LRRD2-4 | N terminus | Present | Fragment | HSA-Linker- |
| | | | (68 a.a.) | Fragment LRRD2 |
| LRRD2-5 | N terminus | Present | Intermediate | HSA-Linker- |
| | | | (130 a.a) | Intermediate LRRD2 |
| LRRD2-6 | N terminus | Present | Full-length | HSA-Linker-Full- |
| | | | (209 a.a) | length LRRD2 |
| LRRD2-7 | C terminus | None | Fragment | Fragment LRRD2- |
| | | | (68 a.a.) | HSA |
| LRRD2-8 | C terminus | None | Intermediate | Intermediate |
| | | | (130 a.a) | LRRD2-HSA |
| LRRD2-9 | C terminus | None | Full-length | Full-length |
| | | | (209 a.a) | LRRD2-HSA |
| LRRD2-10 | C terminus | Present | Fragment | Fragment LRRD2- |
| | | | (68 a.a.) | Linker-HSA |
| LRRD2-11 | C terminus | Present | Intermediate | Intermediate |
| | | | (130 a.a) | LRRD2-Linker- |
| | | | | HSA |
| LRRD2-12 | C terminus | Present | Full-length | Full-length |
| | | | (209 a.a) | LRRD2-Linker- |
| | | | | HSA |

The amino acid sequences of the 12 types of HSA-Slit3LRRD2 fusion proteins are shown in Table 3.

TABLE 3

| Type of | | |
|---|---|---|
| fusion | | SEQ ID |
| protein | Amino acid sequence | NO |
| LRRD2-1 | MMARPLCTLLLLMATLAGALADAHKSEVA | 7 |
| | HRFKDLGEENFKALVLIAFAQYLQQCPFED | |
| | HVKLVNEVTEFAKTCVADESAENCDKSLHT | |
| | LFGDKLCTVATLRETYGEMADCCAKQEPER | |
| | NECFLQHKDDNPNLPRLVRPEVDVMCTAFH | |
| | DNEETFLKKYLYEIARRHPYFYAPELLFFAK | |
| | RYKAAFTECCQAADKAACLLPKLDELRDEG | |
| | KASSAKQRLKCASLQKFGERAFKAWAVAR | |
| | LSQRFPKAEFAEVSKLVTDLTKVHTECCHG | |
| | DLLECADDRADLAKYICENQDSISSKLKECC | |
| | EKPLLEKSHCIAEVENDEMPADLPSLAADFV | |
| | ESKDVCKNYAEAKDVFLGMFLYEYARRHP | |
| | DYSVVLLLRLAKTYETTLEKCCAAADPHEC | |
| | YAKVFDEFKPLVEEPQNLIKQNCELFEQLGE | |
| | YKFQNALLVRYTKKVPQVSTPTLVEVSRNL | |
| | GKVGSKCCKHPEAKRMPCAEDYLSVVLNQ | |
| | LCVLHEKTPVSDRVTKCCTESLVNRRPCFSA | |
| | LEVDETYVPKEFNAETFTFHADICTLSEKER | |
| | QIKKQTALVELVKHKPKATKEQLKAVMDD | |
| | FAAFVEKCCKADDKETCFAEEGKKLVAASQ | |
| | AALGLLTSLVLYGNKITEIAKGLFDGLVSLQ | |
| | LLLLNANKINCLRVNTFQDLQNLNLLSLYD | |
| | NKLQTISKGLFADYKDDDDK | |
| LRRD2-2 | MARPLCTLLLLMATLAGALADAHKSEVAH | 8 |
| | RFKDLGEENFKALVLIAFAQYLQQCPFEDH | |
| | VKLVNEVTEFAKTCVADESAENCDKSLHTL | |
| | FGDKLCTVATLRETYGEMADCCAKQEPER | |
| | NECFLQHKDDNPNLPRLVRPEVDVMCTAFH | |
| | DNEETFLKKYLYEIARRHPYFYAPELLFFAK | |
| | RYKAAFTECCQAADKAACLLPKLDELRDEG | |
| | KASSAKQRLKCASLQKFGERAFKAWAVAR | |
| | LSQRFPKAEFAEVSKLVTDLTKVHTECCHG | |
| | DLLECADDRADLAKYICENQDSISSKLKECC | |
| | EKPLLEKSHCIAEVENDEMPADLPSLAADFV | |
| | ESKDVCKNYAEAKDVFLGMFLYEYARRHP | |
| | DYSVVLLLRLAKTYETTLEKCCAAADPHEC | |
| | YAKVFDEFKPLVEEPQNLIKQNCELFEQLGE | |
| | YKFQNALLVRYTKKVPQVSTPTLVEVSRNL | |
| | GKVGSKCCKHPEAKRMPCAEDYLSVVLNQ | |
| | LCVLHEKTPVSDRVTKCCTESLVNRRPCFSA | |
| | LEVDETYVPKEFNAETFTFHADICTLSEKER | |
| | QIKKQTALVELVKHKPKATKEQLKAVMDD | |
| | FAAFVEKCCKADDKETCFAEEGKKLVAASQ | |
| | AALGLIVEIRLEQNSIKAIPAGAFTQYKKLKR | |
| | IDISKNQISDIAPDAFQGLKSLTSLVLYGNKI | |
| | TEIAKGLFDGLVSLQLLLLNANKINCLRVNT | |
| | FQDLQNLNLLSLYDNKLQTISKGLFAPLQSI | |
| | QTLHLAQNPDYKDDDDK | |
| LRRD2-3 | MARPLCTLLLLMATLAGALADAHKSEVAH | 9 |
| | RFKDLGEENFKALVLIAFAQYLQQCPFEDH | |
| | VKLVNEVTEFAKTCVADESAENCDKSLHTL | |
| | FGDKLCTVATLRETYGEMADCCAKQEPER | |
| | NECFLQHKDDNPNLPRLVRPEVDVMCTAFH | |
| | DNEETFLKKYLYEIARRHPYFYAPELLFFAK | |
| | RYKAAFTECCQAADKAACLLPKLDELRDEG | |
| | KASSAKQRLKCASLQKFGERAFKAWAVAR | |
| | LSQRFPKAEFAEVSKLVTDLTKVHTECCHG | |
| | DLLECADDRADLAKYICENQDSISSKLKECC | |
| | EKPLLEKSHCIAEVENDEMPADLPSLAADFV | |
| | ESKDVCKNYAEAKDVFLGMFLYEYARRHP | |
| | DYSVVLLLRLAKTYETTLEKCCAAADPHEC | |
| | YAKVFDEFKPLVEEPQNLIKQNCELFEQLGE | |
| | YKFQNALLVRYTKKVPQVSTPTLVEVSRNL | |
| | GKVGSKCCKHPEAKRMPCAEDYLSVVLNQ | |
| | LCVLHEKTPVSDRVTKCCTESLVNRRPCFSA | |
| | LEVDETYVPKEFNAETFTFHADICTLSEKER | |
| | QIKKQTALVELVKHKPKATKEQLKAVMDD | |
| | FAAFVEKCCKADDKETCFAEEGKKLVAASQ | |
| | AALGLISCPSPCTCSNNIVDCRGKGLMEIPA | |
| | NLPEGIVEIRLEQNSIKAIPAGAFTQYKKLKR | |
| | IDISKNQISDIAPDAFQGLKSLTSLVLYGNKI | |
| | TEIAKGLFDGLVSLQLLLLNANKINCLRVNT | |
| | FQDLQNLNLLSLYDNKLQTISKGLFAPLQSI | |
| | QTLHLAQNPFVCDCHLKWLADYLQDNPIET | |
| | SGARCSSPRRLANKRISQIKSKKFRCSDYKD | |
| | DDDK | |
| LRRD2-4 | MARPLCTLLLLMATLAGALADAHKSEVAH | 10 |
| | RFKDLGEENFKALVLIAFAQYLQQCPFEDH | |
| | VKLVNEVTEFAKTCVADESAENCDKSLHTL | |
| | FGDKLCTVATLRETYGEMADCCAKQEPER | |
| | NECFLQHKDDNPNLPRLVRPEVDVMCTAFH | |
| | DNEETFLKKYLYEIARRHPYFYAPELLFFAK | |
| | RYKAAFTECCQAADKAACLLPKLDELRDEG | |
| | KASSAKQRLKCASLQKFGERAFKAWAVAR | |
| | LSQRFPKAEFAEVSKLVTDLTKVHTECCHG | |
| | DLLECADDRADLAKYICENQDSISSKLKECC | |
| | EKPLLEKSHCIAEVENDEMPADLPSLAADFV | |
| | ESKDVCKNYAEAKDVFLGMFLYEYARRHP | |
| | DYSVVLLLRLAKTYETTLEKCCAAADPHEC | |
| | YAKVFDEFKPLVEEPQNLIKQNCELFEQLGE | |
| | YKFQNALLVRYTKKVPQVSTPTLVEVSRNL | |
| | GKVGSKCCKHPEAKRMPCAEDYLSVVLNQ | |
| | LCVLHEKTPVSDRVTKCCTESLVNRRPCFSA | |
| | LEVDETYVPKEFNAETFTFHADICTLSEKER | |
| | QIKKQTALVELVKHKPKATKEQLKAVMDD | |
| | FAAFVEKCCKADDKETCFAEEGKKLVAASQ | |
| | AALGLGGGGSGGGGSGGGGSLTSLVLYGN | |
| | KITEIAKGLFDGLVSLQLLLLNANKINCLRV | |
| | NTFQDLQNLNLLSLYDNKLQTISKGLFADY | |
| | KDDDDK | |
| LRRD2-5 | MARPLCTLLLLMATLAGALADAHKSEVAH | 11 |
| | RFKDLGEENFKALVLIAFAQYLQQCPFEDH | |
| | VKLVNEVTEFAKTCVADESAENCDKSLHTL | |
| | FGDKLCTVATLRETYGEMADCCAKQEPER | |
| | NECFLQHKDDNPNLPRLVRPEVDVMCTAFH | |
| | DNEETFLKKYLYEIARRHPYFYAPELLFFAK | |
| | RYKAAFTECCQAADKAACLLPKLDELRDEG | |
| | KASSAKQRLKCASLQKFGERAFKAWAVAR | |
| | LSQRFPKAEFAEVSKLVTDLTKVHTECCHG | |
| | DLLECADDRADLAKYICENQDSISSKLKECC | |
| | EKPLLEKSHCIAEVENDEMPADLPSLAADFV | |
| | ESKDVCKNYAEAKDVFLGMFLYEYARRHP | |
| | DYSVVLLLRLAKTYETTLEKCCAAADPHEC | |
| | YAKVFDEFKPLVEEPQNLIKQNCELFEQLGE | |
| | YKFQNALLVRYTKKVPQVSTPTLVEVSRNL | |
| | GKVGSKCCKHPEAKRMPCAEDYLSVVLNQ | |
| | LCVLHEKTPVSDRVTKCCTESLVNRRPCFSA | |
| | LEVDETYVPKEFNAETFTFHADICTLSEKER | |
| | QIKKQTALVELVKHKPKATKEQLKAVMDD | |
| | FAAFVEKCCKADDKETCFAEEGKKLVAASQ | |
| | AALGLGGGGSGGGGSGGGGSIVEIRLEQNSI | |
| | KAIPAGAFTQYKKLKRIDISKNQISDIAPDAF | |
| | QGLKSLTSLVLYGNKITEIAKGLFDGLVSLQ | |
| | LLLLNANKINCLRVNTFQDLQNLNLLSLYD | |
| | NKLQTISKGLFAPLQSIQTLHLAQNPDYKDD | |
| | DDK | |
| LRRD2-6 | MARPLCTLLLLMATLAGALADAHKSEVAH | 12 |
| | RFKDLGEENFKALVLIAFAQYLQQCPFEDH | |
| | VKLVNEVTEFAKTCVADESAENCDKSLHTL | |
| | FGDKLCTVATLRETYGEMADCCAKQEPER | |
| | NECFLQHKDDNPNLPRLVRPEVDVMCTAFH | |
| | DNEETFLKKYLYEIARRHPYFYAPELLFFAK | |
| | RYKAAFTECCQAADKAACLLPKLDELRDEG | |
| | KASSAKQRLKCASLQKFGERAFKAWAVAR | |
| | LSQRFPKAEFAEVSKLVTDLTKVHTECCHG | |
| | DLLECADDRADLAKYICENQDSISSKLKECC | |
| | EKPLLEKSHCIAEVENDEMPADLPSLAADFV | |
| | ESKDVCKNYAEAKDVFLGMFLYEYARRHP | |
| | DYSVVLLLRLAKTYETTLEKCCAAADPHEC | |
| | YAKVFDEFKPLVEEPQNLIKQNCELFEQLGE | |
| | YKFQNALLVRYTKKVPQVSTPTLVEVSRNL | |
| | GKVGSKCCKHPEAKRMPCAEDYLSVVLNQ | |
| | LCVLHEKTPVSDRVTKCCTESLVNRRPCFSA | |
| | LEVDETYVPKEFNAETFTFHADICTLSEKER | |
| | QIKKQTALVELVKHKPKATKEQLKAVMDD | |
| | FAAFVEKCCKADDKETCFAEEGKKLVAASQ | |
| | AALGLGGGGSGGGGSGGGGSISCPSPCTCSN | |
| | NIVDCRGKGLMEIPANLPEGIVEIRLEQNSIK | |
| | AIPAGAFTQYKKLKRIDISKNQISDIAPDAFQ | |
| | GLKSLTSLVLYGNKITEIAKGLFDGLVSLQL | |
| | LLLNANKINCLRVNTFQDLQNLNLLSLYDN | |
| | KLQTISKGLFAPLQSIQTLHLAQNPFVCDCH | |
| | LKWLADYLQDNPIETSGARCSSPRRLANKRI | |
| | SQIKSKKFRCSDYKDDDDK | |
| LRRD2-7 | MARPLCTLLLLMATLAGALALTSLVLYGNK | 13 |
| | ITEIAKGLFDGLVSLQLLLLNANKINCLRVN | |
| | TFQDLQNLNLLSLYDNKLQTISKGLFADAH | |
| | KSEVAHRFKDLGEENFKALVLIAFAQYLQQ | |
| | CPFEDHVKLVNEVTEFAKTCVADESAENCD | |
| | KSLHTLFGDKLCTVATLRETYGEMADCCAK | |
| | QEPERNECFLQHKDDNPNLPRLVRPEVDVM | |
| | CTAFHDNEETFLKKYLYEIARRHPYFYAPEL | |
| | LFFAKRYKAAFTECCQAADKAACLLPKLDE | |
| | LRDEGKASSAKQRLKCASLQKFGERAFKA | |
| | WAVARLSQRFPKAEFAEVSKLVTDLTKVHT | |
| | ECCHGDLLECADDRADLAKYICENQDSISSK | |
| | LKECCEKPLLEKSHCIAEVENDEMPADLPSL | |
| | AADFVESKDVCKNYAEAKDVFLGMFLYEY | |
| | ARRHPDYSVVLLLRLAKTYETTLEKCCAAA | |
| | DPHECYAKVFDEFKPLVEEPQNLIKQNCELF | |
| | EQLGEYKFQNALLVRYTKKVPQVSTPTLVE | |
| | VSRNLGKVGSKCCKHPEAKRMPCAEDYLS | |
| | VVLNQLCVLHEKTPVSDRVTKCCTESLVNR | |
| | RPCFSALEVDETYVPKEFNAETFTFHADICT | |
| | LSEKERQIKKQTALVELVKHKPKATKEQLK | |
| | AVMDDFAAFVEKCCKADDKETCFAEEGKK | |
| | LVAASQAALGLGGGGSGGGGSGGGGSDY | |
| | KDDDDK | |
| LRRD2-8 | MARPLCTLLLLMATLAGALAIVEIRLEQNSI | 14 |
| | KAIPAGAFTQYKKLKRIDISKNQISDIAPDAF | |
| | QGLKSLTSLVLYGNKITEIAKGLFDGLVSLQ | |
| | LLLLNANKINCLRVNTFQDLQNLNLLSLYD | |
| | NKLQTISKGLFAPLQSIQTLHLAQNPDAHKS | |
| | EVAHRFKDLGEENFKALVLIAFAQYLQQCP | |
| | FEDHVKLVNEVTEFAKTCVADESAENCDKS | |
| | LHTLFGDKLCTVATLRETYGEMADCCAKQ | |
| | EPERNECFLQHKDDNPNLPRLVRPEVDVMC | |
| | TAFHDNEETFLKKYLYEIARRHPYFYAPELL | |
| | FFAKRYKAAFTECCQAADKAACLLPKLDEL | |
| | RDEGKASSAKQRLKCASLQKFGERAFKAW | |
| | AVARLSQRFPKAEFAEVSKLVTDLTKVHTE | |
| | CCHGDLLECADDRADLAKYICENQDSISSKL | |
| | KECCEKPLLEKSHCIAEVENDEMPADLPSLA | |
| | ADFVESKDVCKNYAEAKDVFLGMFLYEYA | |
| | RRHPDYSVVLLLRLAKTYETTLEKCCAAAD | |
| | PHECYAKVFDEFKPLVEEPQNLIKQNCELFE | |
| | QLGEYKFQNALLVRYTKKVPQVSTPTLVEV | |
| | SRNLGKVGSKCCKHPEAKRMPCAEDYLSV | |
| | VLNQLCVLHEKTPVSDRVTKCCTESLVNRR | |
| | PCFSALEVDETYVPKEFNAETFTFHADICTLS | |
| | EKERQIKKQTALVELVKHKPKATKEQLKAV | |
| | MDDFAAFVEKCCKADDKETCFAEEGKKLV | |
| | AASQAALGLGGGGSGGGGSGGGGSDYKD | |
| | DDDK | |
| LRRD2-9 | MARPLCTLLLLMATLAGALAISCPSPCTCSN | 15 |
| | NIVDCRGKGLMEIPANLPEGIVEIRLEQNSIK | |
| | AIPAGAFTQYKKLKRIDISKNQISDIAPDAFQ | |
| | GLKSLTSLVLYGNKITEIAKGLFDGLVSLQL | |
| | LLLNANKINCLRVNTFQDLQNLNLLSLYDN | |
| | KLQTISKGLFAPLQSIQTLHLAQNPFVCDCH | |
| | LKWLADYLQDNPIETSGARCSSPRRLANKRI | |
| | SQIKSKKFRCSDAHKSEVAHRFKDLGEENF | |
| | KALVLIAFAQYLQQCPFEDHVKLVNEVTEF | |
| | AKTCVADESAENCDKSLHTLFGDKLCTVAT | |
| | LRETYGEMADCCAKQEPERNECFLQHKDD | |
| | NPNLPRLVRPEVDVMCTAFHDNEETFLKKY | |
| | LYEIARRHPYFYAPELLFFAKRYKAAFTECC | |
| | QAADKAACLLPKLDELRDEGKASSAKQRL | |
| | KCASLQKFGERAFKAWAVARLSQRFPKAEF | |
| | AEVSKLVTDLTKVHTECCHGDLLECADDRA | |
| | DLAKYICENQDSISSKLKECCEKPLLEKSHCI | |
| | AEVENDEMPADLPSLAADFVESKDVCKNY | |
| | AEAKDVFLGMFLYEYARRHPDYSVVLLLRL | |
| | AKTYETTLEKCCAAADPHECYAKVFDEFKP | |
| | LVEEPQNLIKQNCELFEQLGEYKFQNALLVR | |
| | YTKKVPQVSTPTLVEVSRNLGKVGSKCCKH | |
| | PEAKRMPCAEDYLSVVLNQLCVLHEKTPVS | |
| | DRVTKCCTESLVNRRPCFSALEVDETYVPK | |
| | EFNAETFTFHADICTLSEKERQIKKQTALVE | |
| | LVKHKPKATKEQLKAVMDDFAAFVEKCCK | |
| | ADDKETCFAEEGKKLVAASQAALGLGGGG | |
| | SGGGGSGGGGSDYKDDDDK | |
| LRRD2-10 | MARPLCTLLLLMATLAGALALTSLVLYGNK | 16 |
| | ITEIAKGLFDGLVSLQLLLLNANKINCLRVN | |
| | TFQDLQNLNLLSLYDNKLQTISKGLFAGGG | |
| | GSGGGGSGGGGSDAHKSEVAHRFKDLGEE | |
| | NFKALVLIAFAQYLQQCPFEDHVKLVNEVT | |
| | EFAKTCVADESAENCDKSLHTLFGDKLCTV | |
| | ATLRETYGEMADCCAKQEPERNECFLQHK | |
| | DDNPNLPRLVRPEVDVMCTAFHDNEETFLK | |
| | KYLYEIARRHPYFYAPELLFFAKRYKAAFTE | |
| | CCQAADKAACLLPKLDELRDEGKASSAKQ | |
| | RLKCASLQKFGERAFKAWAVARLSQRFPKA | |
| | EFAEVSKLVTDLTKVHTECCHGDLLECADD | |
| | RADLAKYICENQDSISSKLKECCEKPLLEKS | |
| | HCIAEVENDEMPADLPSLAADFVESKDVCK | |
| | NYAEAKDVFLGMFLYEYARRHPDYSVVLL | |
| | LRLAKTYETTLEKCCAAADPHECYAKVFDE | |
| | FKPLVEEPQNLIKQNCELFEQLGEYKFQNAL | |
| | LVRYTKKVPQVSTPTLVEVSRNLGKVGSKC | |
| | CKHPEAKRMPCAEDYLSVVLNQLCVLHEK | |
| | TPVSDRVTKCCTESLVNRRPCFSALEVDETY | |
| | VPKEFNAETFTFHADICTLSEKERQIKKQTA | |
| | LVELVKHKPKATKEQLKAVMDDFAAFVEK | |
| | CCKADDKETCFAEEGKKLVAASQAALGLG | |
| | GGGSGGGGSGGGGSDYKDDDDK | |
| LRRD2-11 | MARPLCTLLLLMATLAGALAIVEIRLEQNSI | 17 |
| | KAIPAGAFTQYKKLKRIDISKNQISDIAPDAF | |
| | QGLKSLTSLVLYGNKITEIAKGLFDGLVSLQ | |
| | LLLLNANKINCLRVNTFQDLQNLNLLSLYD | |
| | NKLQTISKGLFAPLQSIQTLHLAQNPGGGGS | |
| | GGGGSGGGGSDAHKSEVAHRFKDLGEENF | |
| | KALVLIAFAQYLQQCPFEDHVKLVNEVTEF | |
| | AKTCVADESAENCDKSLHTLFGDKLCTVAT | |
| | LRETYGEMADCCAKQEPERNECFLQHKDD | |
| | NPNLPRLVRPEVDVMCTAFHDNEETFLKKY | |
| | LYEIARRHPYFYAPELLFFAKRYKAAFTECC | |
| | QAADKAACLLPKLDELRDEGKASSAKQRL | |
| | KCASLQKFGERAFKAWAVARLSQRFPKAEF | |
| | AEVSKLVTDLTKVHTECCHGDLLECADDRA | |
| | DLAKYICENQDSISSKLKECCEKPLLEKSHCI | |
| | AEVENDEMPADLPSLAADFVESKDVCKNY | |
| | AEAKDVFLGMFLYEYARRHPDYSVVLLLRL | |
| | AKTYETTLEKCCAAADPHECYAKVFDEFKP | |
| | LVEEPQNLIKQNCELFEQLGEYKFQNALLVR | |
| | YTKKVPQVSTPTLVEVSRNLGKVGSKCCKH | |
| | PEAKRMPCAEDYLSVVLNQLCVLHEKTPVS | |
| | DRVTKCCTESLVNRRPCFSALEVDETYVPK | |
| | EFNAETFTFHADICTLSEKERQIKKQTALVE | |
| | LVKHKPKATKEQLKAVMDDFAAFVEKCCK | |
| | ADDKETCFAEEGKKLVAASQAALGLGGGG | |
| | SGGGGSGGGGSDYKDDDDK | |
| LRRD2-12 | MARPLCTLLLLMATLAGALAISCPSPCTCSN | 18 |
| | NIVDCRGKGLMEIPANLPEGIVEIRLEQNSIK | |
| | AIPAGAFTQYKKLKRIDISKNQISDIAPDAFQ | |
| | GLKSLTSLVLYGNKITEIAKGLFDGLVSLQL | |
| | LLLNANKINCLRVNTFQDLQNLNLLSLYDN | |
| | KLQTISKGLFAPLQSIQTLHLAQNPFVCDCH | |
| | LKWLADYLQDNPIETSGARCSSPRRLANKRI | |
| | SQIKSKKFRCSGGGGSGGGGSGGGGSDAHK | |
| | SEVAHRFKDLGEENFKALVLIAFAQYLQQC | |
| | PFEDHVKLVNEVTEFAKTCVADESAENCDK | |
| | SLHTLFGDKLCTVATLRETYGEMADCCAKQ | |
| | EPERNECFLQHKDDNPNLPRLVRPEVDVMC | |
| | TAFHDNEETFLKKYLYEIARRHPYFYAPELL | |
| | FFAKRYKAAFTECCQAADKAACLLPKLDEL | |
| | RDEGKASSAKQRLKCASLQKFGERAFKAW | |
| | AVARLSQRFPKAEFAEVSKLVTDLTKVHTE | |
| | CCHGDLLECADDRADLAKYICENQDSISSKL | |
| | KECCEKPLLEKSHCIAEVENDEMPADLPSLA | |
| | ADFVESKDVCKNYAEAKDVFLGMFLYEYA | |
| | RRHPDYSVVLLLRLAKTYETTLEKCCAAAD | |
| | PHECYAKVFDEFKPLVEEPQNLIKQNCELFE | |
| | QLGEYKFQNALLVRYTKKVPQVSTPTLVEV | |
| | SRNLGKVGSKCCKHPEAKRMPCAEDYLSV | |
| | VLNQLCVLHEKTPVSDRVTKCCTESLVNRR | |
| | PCFSALEVDETYVPKEFNAETFTFHADICTLS | |
| | EKERQIKKQTALVELVKHKPKATKEQLKAV | |
| | MDDFAAFVEKCCKADDKETCFAEEGKKLV | |
| | AASQAALGLGGGGSGGGGSGGGGDYKD | |
| | DDDK | |

*The underlined sequence in Table 3 is a GS linker linking HSA and LRRD2, and the bold sequence is a GS linker linking a sequence added to the C-terminus in order to express the fusion protein in its final form.

2-2. Confirmation of Receptor Binding Ability of Various Forms of HSA-Slit3 LRRD2 Fusion Proteins

Slit3 LRRD2 binds to the Robo1 or Robo2 receptor, and as a result, the (3-catenin binding to the M-cadherin of myoblasts is released via the Slit-Robo system to activate the β-catenin and increase the expression of myogenin, and subsequently, the formation of muscles is promoted by inducing the differentiation of myoblasts. Therefore, in the present example, the receptor binding ability of the 12 types of HSA-Slit3 LRRD2 fusion proteins prepared in Example 2-1 was confirmed. The binding ability of 12 types of HSA-Slit3 LRRD2 fusion proteins to the Robo1 receptor was quantified using an ELISA system. Detailed conditions are as follows.

96-well microtiter plates (manufactured by NUNC) were coated with 12 types of HSA-Slit3 LRRD2 fusion proteins at 4° C. for 18 hours at 0, 1, 10, 100, and 1000 nM per well, in consideration of the molecular weight. The coated material was washed three times using PBS containing 0.05% Tween 20 (PBST). Blocking was performed with PBST supplemented with 1% BSA at room temperature for 2 hours to block non-specific binding. The coated material was washed three times with PBST to remove a blocking buffer. After washing, 30 ug of a protein obtained from the corresponding osteoblastic cell line was allowed to adhere (lysis buffer: 0.5% NP40, 50 mM Tris pH 7.5, 150 mM NaCl, 1 mM EDTA, 0.2 mM NaF, 1 mM Na3VO4, 1 mM DTT, 1 mM PMSF, and a proteinase inhibitor cocktail) at room temperature for 2 hours. After washing three times with PBST, a Robo1 antibody (abcam: ab7279) diluted with 0.1% BSA at 1:1000 was adhered thereto at room temperature for 2 hours. After washing three times with PBST, an HRP-binding antibody (cell signaling: 7074) diluted with 0.1% BSA at 1:2000 was adhered thereto at room temperature for 2 hours. After washing five times with PBST, a reaction was performed with a TMB solution at 37° C. for 30 minutes. To stop the reaction, 100 μl of 1 N H₂SO₄ was used, and absorbance was measured at 450 nm.

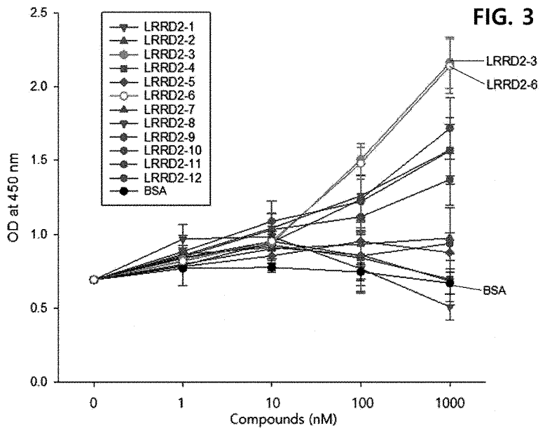

As a result, as illustrated in FIG. 3, it was confirmed that the receptor binding ability of LRRD2-3 and LRRD2-6 was the best.

### Example 3

Pharmacokinetic Studies of Slit3 LRRD2 and HSA-Slit3 LRRD2 Fusion Proteins in Mice

In the present example, based on the results of Example 2, a pharmacokinetic study was conducted by selecting LRRD2-3, which has the best receptor binding ability.

A pharmacokinetic study is a part of new drug development processes, and aims to obtain information on the absorption, distribution, metabolism and excretion of a test drug by assessing changes in drug concentration in the body over time. In the present example, pharmacokinetic properties were confirmed in mice after a single intravenous administration of Slit3 LRRD2-3 and HSA-Slit3 LRRD2 fusion protein (LRRD2-3).

3-1. Chemicals and Solvents

The carbamazepine used in this example was purchased from Sigma Aldrich, and HPLC grade acetonitrile and methanol were purchased from J. T. Baker.

3-2. Animals and Administration Conditions

In the present example, ICR-based male mice (6 weeks old, Orient Bio Co., Ltd., Seongnam, Republic of Korea) with a body weight ranging from 30 to 32.5 g were used. Mice were fasted for 4 hours before the experiment and fasting was maintained for up to 4 hours after administration. The breeding place was given 12 hours each of light and dark, and an appropriate temperature (20 to 25° C.) and humidity (40 to 60%) were maintained.

TABLE 4
Pharmacokinetic test

| Administered | Number of | Administration |
|---|---|---|
| material | animals | dose |
| Slit3 LRRD2 | 4 | 10 mg/kg |
| HSA-Slit3 LRRD2 | 3 | 35 mg/kg |
| (LRRD2-3) | | |
| Total | 7 | — |

Slit3 LRRD2 was prepared by being dissolved in PBS at a dose of 1 mg/mL. HSA-Slit3 LRRD2 (LRRD2-3) was prepared by being dissolved in PBS at a dose of 3.5 mg/mL (1 mg/mL for Slit3 LRRD2) in consideration of the molecular weight. The dose was 10 mL/kg in both groups, and the prepared solution was administered through the left caudal vein.

3-3. Pharmacokinetic Test

In the case of the pharmacokinetic test, fasted mice were administered Slit3 LRRD2 and HSA-Slit3 LRRD2 (LRRD2-3) at a dose of 10 mg/kg and 35 mg/kg, respectively, through the caudal vein. After administration, mice were fixed by hand at 0.05, 0.12, 0.33, 1, 3, 7, 10, 24, 48, and 72 hours, respectively, and then 70 μL of blood was collected from the right orbital venous plexus using heparin-coated capillary tubes. The collected blood was centrifuged for 5 minutes and then stored frozen at −20° C. until plasma was isolated and analyzed.

3-4. Analysis Method

The concentration of Slit3 LRRD2 in plasma samples was quantified using an HPLC/MS/MS system. Before sample pretreatment, plasma samples were purified using Ni-NTA magnetic beads. After purified Slit3 LRRD2 and HSA-Slit3 LRRD2 (LRRD2-3) were denatured by adding 6M urea and 18 mM dithiothreitol (DTT) thereto, alkylation was induced using 225 mM iodine acetamide. Then, to obtain a signature peptide, 850 ng of recombinant porcine trypsin (V5117, Promega, Madison, Wis., USA) was added thereto, and the resulting mixture was reacted in a water bath set at 37° C. for 24 hours. After 50 μL of 3% formic acid dissolved in MeOH was added to 70 μL of a trypsin digestion product produced after the reaction, the mixed sample was suspended using a vortex mixer for 10 minutes, centrifuged at 13,500 rpm for 10 minutes, and 160 μL of the supernatant was taken and transferred to an analysis vessel, and 5 μL of the transferred supernatant was injected into an HPLC MSMS system to perform analysis.

Detailed analysis conditions are as follows.
HPLC system: Agilent 1100 (Agilent Technologies, Santa Clara, Calif.)
Column: ZORBAX® C₈ 3.5 μm, 2.1*50 mm (Agilent)
Mobile phase:
   A: 0.1% formic acid dissolved in distilled water
   B: Acetonitrile
   (Isocratic elution)

| | |
|---|---|
| Time | 0 → 0.1 → 1.0 → 1.5 → 2.5 → 3 → 5 |
| B (%) | 5 → 5 → 5 → 95 → 95 → 5 → 5 |

Flow rate: 300 μL/min—Temperature: 20° C. in column, and 10° C. in autosampler tray
Runtime: 5 minutes
Detection: Tandem quadrupole mass spectrometer (API 4000, QTRAP®, Applied Biosystems/MDS SCIEX, Foster City, Calif., USA)
Curtain gas: 20 psi
Ion source gas 1: 50 psi
Ion source gas 2: 60 psi
Ionspray voltage: 5500 V
Temperature: 600° C.
Multiple-reaction-monitoring (MRM) mode: Positive

The molecular ions of a Silt3 LRRD2 signature peptide (P6) were fragmented by a collision energy of 23 V, and a collision gas was set to ‘medium (8 psi)’ in the equipment. Ions were detected in the ESI-positive MRM mode, and P6 was quantified from 587.97 to 491.50 in units of m/z. Detected peaks were integrated using Analyst software version 1.4.2 (Applied Biosystems/MDS SCIEX). A quantifiable range of Silt3 LRRD2 in plasma was 1 to 100 μg/mL, and that of HSA-Silt3 LRRD2 (LRRD2-3) was 3 to 100 μg/mL. In the corresponding analysis, Slit3 LRRD2 showed a peak retention time of 3.29 minutes.

3-5. Data Analysis

The concentration of CNC00000 in plasma over time was determined using the LC-MS/MS analysis method described in Example 3-4, and pharmacokinetic parameters (PK parameters) were calculated using a non-compartmental analysis of WinNonlin® 4.2 (Pharsight Corp., Cary, N.C., USA) software. The maximum concentration (Cₘₐₓ) and the maximum concentration arrival time (Tₘₐₓ) were temporally calculated from a curve according to the blood drug concentration vs. time, and the elimination rate constant (Kₑ) was calculated by a linear regression analysis in the terminal phase of the log scale. The half-life (T_{1/2}) was calculated by dividing LN2 by Ke, and an area under the curve of blood drug concentration vs. time (AUC_{0-∞}) and an area under the curve of blood drug moment vs. time (AUMC_{0-∞}) were calculated by the linear trapezoidal rule and the standard area extrapolation method. Clearance (CL) and steady state volume of distribution (Vss) were calculated by the following [Equation 1] to [Equation 3]:

$$\begin{matrix}CL = \frac{Dose}{{AUC}_{0 - ∞}} & [ Equation 1 ] \\ {V}_{ss} = MRT \times CL & [ Equation 2 ] \\ MRT = \frac{{AUMC}_{0 - ∞}}{{AUC}_{0 - ∞}} & [ Equation 3 ]\end{matrix}$$

3-6. Results

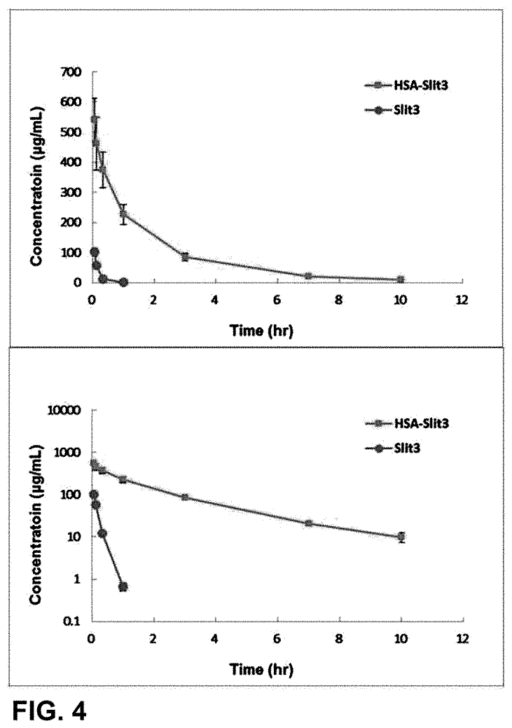

The concentrations of Slit3 LRRD2 and HSA-Slit3 LRRD2 (LRRD2-3) in plasma over time are shown in FIG. 4 and Tables 5 and 6, and pharmacokinetic parameters are shown in Table 6. The related parameters and all values were calculated for each individual and then averaged. Referring to the blood concentration pattern and animal experiment record over time, any abnormal populations were excluded from the data analysis, and the experimental group used for data analysis was set to have at least n=3 or more.

TABLE 5
Plasma concentration after intravenous administration of Slit3
Plasma concentration of Slit3 (μg/mL)

| Time (h) | #1 | #2 | #3 | #4 | mean | S.D. |
|---|---|---|---|---|---|---|
| 0.05 | 122 | 90.9 | 97.6 | 96.7 | 102 | 13.8 |
| 0.12 | 62.6 | 61.4 | 47.9 | 59.7 | 57.9 | 6.77 |
| 0.33 | 14.1 | 11.4 | 12.1 | 11.0 | 12.2 | 1.38 |
| 1 | 0.66 | 0.48 | 0.84 | 0.71 | 0.67 | 0.15 |
| 3 | BQL | BQL | BQL | BQL | 0.000 | — |
| 7 | BQL | BQL | BQL | BQL | 0.000 | — |
| 10 | BQL | BQL | BQL | BQL | 0.000 | — |
| 24 | BQL | BQL | BQL | BQL | 0.000 | — |

*BQL: When it is less than the quantification limit, it is treated as “0”.

TABLE 6
Plasma concentration after intravenous
administration of HSA-Slit3 (LRRD2-3)
Plasma concentration of Slit3 (μg/mL)

| Time (h) | #6 | #7 | #8 | mean | S.D. |
|---|---|---|---|---|---|
| 0.05 | 587 | 460 | 577 | 541 | 70.6 |
| 0.12 | 539 | 366 | 480 | 462 | 87.9 |
| 0.33 | 355 | 327 | 441 | 374 | 59.4 |
| 1 | 217 | 199 | 262 | 226 | 32.4 |
| 3 | 82.8 | 73.6 | 98.9 | 85.1 | 12.8 |
| 7 | 18.9 | 18.5 | 23.4 | 20.3 | 2.72 |
| 10 | 9.71 | 7.64 | 12.5 | 9.95 | 2.44 |
| 24 | BQL | BQL | BQL | 0.000 | — |

*BQL: When it is less than the quantification limit, it is treated as “0”.

As confirmed in the following Table 7, the HSA-bound Slit3 LRRD2 (LRRD2-3) showed an approximately 14-fold improved half-life compared to Slit3 LRRD2.

TABLE 7

| | | | | HSA-Slit3 LRRD2 | | |
|---|---|---|---|---|---|---|
| | Slit3 LRRD2 | | | (LRRD2-3) | | |
| Parameter | Average | | S.D. | Average | | S.D. |
| Tₘₐₓ (h) | 0.050 | ± | 0.000 | 0.050 | ± | 0.000 |
| C₀ (μg/mL) | 153.9 | | 33.25 | 607.8 | | 59.87 |
| Cₘₐₓ (μg/mL) | 101.8 | ± | 13.79 | 541.3 | ± | 70.61 |
| T_{1/2} (h) | 0.139 | ± | 0.012 | 1.993 | ± | 0.147 |
| AUCₐₗₗ (μg · h/mL) | 23.63 | ± | 2.534 | 919.9 | ± | 131.2 |
| AUC_{inf} (μg · h/mL) | 23.77 | ± | 2.530 | 948.1 | ± | 136.1 |
| CL (mL/h/kg) | 424.0 | ± | 40.88 | 10.69 | ± | 1.504 |
| Vₛₛ (mL/kg) | 61.46 | ± | 7.368 | 24.1 | ± | 3.293 |

### Example 4

Confirmation of In Vivo Efficacy of HSA-Bound Slit3 LRRD2

9-week-old Balbc-nude mice subjected to an ovariectomy were treated with Slit3 LRRD2 to which albumin was not bound or HSA-Slit3 LRD2 fusion protein (LRRD2-3) for 4 weeks from the time when the mice became 11 weeks old. Each drug was administered by intravenous injection once daily, five times per week, and Slit3 LRRD2 and HSA-bound Slit3 LRRD2 (LRRD2-3) were injected daily in a dose of 10 mg and 37.13 mg, respectively (Slit3 LRRD2 corresponds to 10 mg daily). After the administration was completed, the soleus muscle was collected and the weight of the muscle was measured, and the results are shown in the following Table 8.

TABLE 8

| | Muscle mass (mg) |
|---|---|
| Group | of soleus muscle |
| Control (n = 8) | 5.89 ± 0.25 |
| Slit3 LRRD2 (n = 8) | 6.73 ± 0.20* |
| HSA-Slit3 LRRD2 fusion protein | 6.80 ± 1.75* |
| (LRRD2-3) (n = 8) | |

*P < 0.05, vs. non-treated control

As shown in Table 8, both albumin-unbound Slit3 LRRD2 and HSA-bound Slit3 LRRD2 significantly increased the muscle mass of soleus muscle. However, HSA-bound Slit3 LRRD2 showed even stronger therapeutic efficacy than albumin-unbound Slit3 LRRD2.

### INDUSTRIAL APPLICABILITY

Since the albumin-bound LRRD2 of the Slit3 protein exhibits the same cytological efficacy as albumin-unbound LRRD2 of the Slit3 protein and has a significantly increased in vivo half-life compared to albumin-unbound LRRD2 of the Slit3 protein, bone-related diseases can be more effectively prevented or treated.

The national research and development projects supporting the present invention are as follows.

(1) [National Research and Development Projects Supporting the Present Invention]

[Project Identification Number] 2017-1229 (HI15C0377010017)

[Ministry Name] Ministry of Health and Welfare

[Research Management Agency] Korea Health Industry Development Institute

[Research Project Name] Disease Oriented Translational Research

[Research Title] Discovery of macronuclear cell secretion factors with bone formation promotion

[Contribution Rate] 75/100

[Administrative Organization] Asan Medical Center, Seoul

[Research Period] Sep. 7, 2017 to Sep. 6, 2018

(2) [National Research and Development Projects Supporting the Present

Invention]

[Project Identification Number] 2013-2234 (HIT3C1634060018)

[Ministry Name] Ministry of Health and Welfare

[Research Management Agency] Korea Health Industry Development Institute

[Research Project Name] Disease Oriented Translational Research

[Research Title] Pharmacokinetic Study of Slit3 LRRD2 and in vivo Toxicity Verification Using Slit3 TG Mice

[Contribution Rate] 25/100

[Administrative Organization] Industry & Academic Cooperation in Chungnam National University (IAC)

[Research Period] Nov. 1, 2013 to Jun. 30, 2019

1. A fusion protein comprising albumin-bound LRRD2 of the Slit3 protein.

2. The fusion protein of claim 1, wherein the albumin is human serum albumin.

3. The fusion protein of claim 2, wherein the human serum albumin is bound to the N-terminus of the LRRD2 of the Slit3 protein.

4. The fusion protein of claim 3, wherein the human serum albumin comprises an amino acid sequence of SEQ ID NO: 2, and the LRRD2 of the Slit3 protein comprises an amino acid sequence of SEQ ID NO: 3.

5. The fusion protein of claim 1, further comprising a linker between the albumin and the LRRD2 of the Slit3 protein.

6. The fusion protein of claim 5, wherein the linker is (GGGGS)n, wherein n is an integer from 1 to 10.

7. A nucleic acid molecule encoding the fusion protein of claim 1.

8. A recombinant vector comprising the nucleic acid molecule of claim 7.

9. A transformant comprising the recombinant vector of claim 8.

10. A method for preparing a fusion protein comprising albumin-bound LRRD2 of the Slit3 protein, the method comprising culturing the transformant of claim 9.

11. A pharmaceutical composition comprising the fusion protein of claim 1.

12. The pharmaceutical composition of claim 11, wherein the pharmaceutical composition is administered as an injection.

13. The pharmaceutical composition of claim 11, which is for use in prevention or treatment of a muscle disease.

14. The pharmaceutical composition of claim 13, wherein the muscle disease is one or more selected from the group consisting of atony, muscular atrophy, muscular dystrophy, muscle degeneration, myasthenia gravis, cachexia, and sarcopenia.

15. A method of preventing or treating a muscle disease, comprising administering to a subject in need thereof a therapeutically effective amount of the fusion protein of claim 1.

16. The method of claim 15, wherein the pharmaceutical composition is administered as an injection.

17. The method of claim 15, wherein the muscle disease is one or more selected from the group consisting of atony, muscular atrophy, muscular dystrophy, muscle degeneration, myasthenia gravis, cachexia, and sarcopenia.

18. A method of improving the in vivo half-life of LRRD2 of the Slit3 protein using the fusion protein of claim 1.


SEQUENCE LISTING (18 sequences)

SEQ ID NO: 1 - protein, 20 aa, Artificial
  SP Cystatin S
MARPLCTLLLLMATLAGALA

SEQ ID NO: 2 - protein, 585 aa, Artificial [sequence omitted]

SEQ ID NO: 3 - protein, 209 aa, Artificial
  Slit3 LRRD2
ISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCS

SEQ ID NO: 4 - protein, 8 aa, Artificial
  FLAG
DYKDDDDK

SEQ ID NO: 5 - protein, 5 aa, Artificial
  GS linker
  REPEAT (1)..(5): The amino acid residues at Position 1 to Postion 5 are repeated 1 to 10 times.
GGGGS

SEQ ID NO: 6 - protein, 15 aa, Artificial
  GS linker
GGGGSGGGGSGGGGS

SEQ ID NO: 7 - protein, 681 aa, Artificial
  LRRD2-1
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLLTSLVLYGNKITEIAKGLFDGLVSLQLLLLNANKINCLRVNTFQDLQNLNLLSLYDNKLQTISKGLFADYKDDDDK

SEQ ID NO: 8 - protein, 743 aa, Artificial
  LRRD2-2
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPDYKDDDDK

SEQ ID NO: 9 - protein, 822 aa, Artificial
  LRRD2-3
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSDYKDDDDK

SEQ ID NO: 10 - protein, 696 aa, Artificial
  LRRD2-4
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSLTSLVLYGNKITEIAKGLFDGLVSLQLLLLNANKINCLRVNTFQDLQNLNLLSLYDNKLQTISKGLFADYKDDDDK

SEQ ID NO: 11 - protein, 758 aa, Artificial
  LRRD2-5
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPDYKDDDDK

SEQ ID NO: 12 - protein, 837 aa, Artificial
  LRRD2-6
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSDYKDDDDK

SEQ ID NO: 13 - protein, 696 aa, Artificial
  LRRD2-7
MARPLCTLLLLMATLAGALALTSLVLYGNKITEIAKGLFDGLVSLQLLLLNANKINCLRVNTFQDLQNLNLLSLYDNKLQTISKGLFADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 14 - protein, 758 aa, Artificial
  LRRD2-8
MARPLCTLLLLMATLAGALAIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 15 - protein, 837 aa, Artificial
  LRRD2-9
MARPLCTLLLLMATLAGALAISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 16 - protein, 711 aa, Artificial
  LRRD2-10
MARPLCTLLLLMATLAGALALTSLVLYGNKITEIAKGLFDGLVSLQLLLLNANKINCLRVNTFQDLQNLNLLSLYDNKLQTISKGLFAGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 17 - protein, 773 aa, Artificial
  LRRD2-11
MARPLCTLLLLMATLAGALAIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 18 - protein, 852 aa, Artificial
  LRRD2-12
MARPLCTLLLLMATLAGALAISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK